(19) United States
(12) Patent Application Publication
(10) Pub. No.: US 2022/0218290 A1
(43) Pub. Date: Jul. 14, 2022

(54) SYSTEMS AND METHODS CONFIGURED TO PROVIDE ASTHMA-RELATED INFORMATION OF SUBJECTS BASED ON ENVIRONMENTAL CONDITIONS AND/OR SUBJECT CONDITION INFORMATION

(71) Applicant: AsthmaTek, Inc., Danville, CA (US)

(72) Inventor: Jemal Dean Zikria, Danville, CA (US)

(21) Appl. No.: 17/148,329

(22) Filed: Jan. 13, 2021

(51) Int. Cl. A61B 5/00; G16H 40/67; G16H 10/60; G16H 50/30; G16H 10/20; A61B 5/0205

(52) U.S. Cl. CPC A61B 5/7282; G16H 40/67; G16H 10/60; G16H 50/30; A61B 5/1112; A61B 5/742; A61B 5/7475; A61B 5/0205; A61B 5/486; G16H 10/20

(57) ABSTRACT

Systems and methods configured to provide asthma-related information of subjects based on environmental conditions and/or subject condition information are disclosed. Exemplary implementations may: obtain location information related to a physical location of a subject over time; obtain, based on the location information, environmental conditions at or around the physical location indicated by the location information; obtain subject condition information defining remote diagnostic information and subject health information; and effectuate presentation of a graphical user interface that conveys the subject condition information.

### FIELD OF THE DISCLOSURE

The present disclosure relates to systems and methods configured to provide asthma-related information of subjects based on environmental conditions and/or subject condition information.

### BACKGROUND

Individuals with asthma may only monitor and treat for his/her asthma symptoms, conditions, and causes. However, the individuals often do not monitor their conditions and causes correctly nor treat their symptoms correctly without information directly from a physician.

### SUMMARY

One aspect of the present disclosure relates to a system configured to obtain information relevant to asthma of a subject and present the obtained information to the subject. Location information indicative of a physical location of the subject may be obtained to facilitate obtainment of environmental conditions at the physical location. The environmental conditions may affect the asthma of the subject. Furthermore, subject condition information that includes results to at least a remote fractional exhaled nitric oxide (FeNO) test, other tests, and subject health information of the subject are obtained. A graphical user interface may be presented so that the information obtained may be presented to the subject. Thus, the subject may have easy immediate access to the information to correctly manage and treat their asthma.

Another aspect of the present disclosure relates to a system configured to provide a respiratory health score of the subject. The respiratory health score may indicate a current respiratory condition of the subject relative to the asthma of the subject. Similar to the other aspect of the present disclosure, location information, environmental conditions based on the location information, subject condition information, and physician-certified condition information may be obtained. The physician-certified condition information may include at least results to a physician-administered FeNO test of the subject. The respiratory health score may be based on the environmental conditions, the subject condition information, and the physician-certified condition information obtained. Thus, the subject and/or a physician may be provided the current respiratory condition of the subject relative to their asthma to better manage and treat their asthma based on such.

One aspect of the present disclosure relates to a system configured to provide asthma-related information of subjects based on environmental conditions and/or subject condition information. The system may include one or more hardware processors configured by machine-readable instructions. Instruction components may include one or more of environmental conditions component, subject condition component, user interface effectuation component, and/or other instruction components.

The environmental conditions component may be configured to obtain location information related to a physical location of a subject over time. The location information may indicate the physical location of the subject. The environmental conditions component may be configured to obtain, based on the location information, environmental conditions at or around the physical location indicated by the location information.

The subject condition component may be configured to obtain subject condition information and/or other information. The subject condition information may define remote diagnostic information, subject health information, and/or other information. The remote diagnostic information may include results of a FeNO test and/or other remote tests.

The user interface effectuation component may be configured to effectuate presentation of a graphical user interface that conveys at least the subject condition information and other information related to the subject.

Another aspect of the present disclosure relates to a system configured to provide a respiratory health score of a subject. This system may include one or more other hardware processors configured by other machine-readable instructions. Instruction components for this system may include instruction components similar to the environmental conditions component, the subject condition component, and a physician-certified information component, a score determination component, and/or other instruction components.

The physician-certified information component may be configured to obtain physician-certified condition information. The physician-certified condition information may include at least physician-supervised diagnostic information and other information. The physician-supervised diagnostic information may include results of a physician-administered FeNO test and/or other tests.

The score determination component may be configured to determine the respiratory health score of the subject based on the environmental conditions, the subject condition information, the physician-certified condition information, and/or other information. The respiratory health score may indicate a current respiratory condition of the subject relative to asthma of the subject.

As used herein, the term “obtain” (and derivatives thereof) may include active and/or passive retrieval, determination, derivation, transfer, upload, download, submission, and/or exchange of information, and/or any combination thereof. As used herein, the term “effectuate” (and derivatives thereof) may include active and/or passive causation of any effect, both local and remote. As used herein, the term “determine” (and derivatives thereof) may include measure, calculate, compute, estimate, approximate, generate, and/or otherwise derive, and/or any combination thereof.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

FIG. 1A illustrates a system configured to provide asthma-related information of subjects based on environmental conditions and/or subject condition information, in accordance with one or more implementations.

FIG. 1B illustrates a system configured to provide a respiratory health score of a subject, in accordance with one or more implementations.

FIG. 2A illustrates a method to provide asthma-related information of subjects based on environmental conditions and/or subject condition information, in accordance with one or more implementations.

FIG. 2B illustrates a method to provide a respiratory health score of a subject, in accordance with one or more implementations.

FIG. 3A-D illustrate example implementations of the system configured to provide asthma-related information of subjects based on environmental conditions and/or subject condition information, in accordance with one or more implementations.

### DETAILED DESCRIPTION

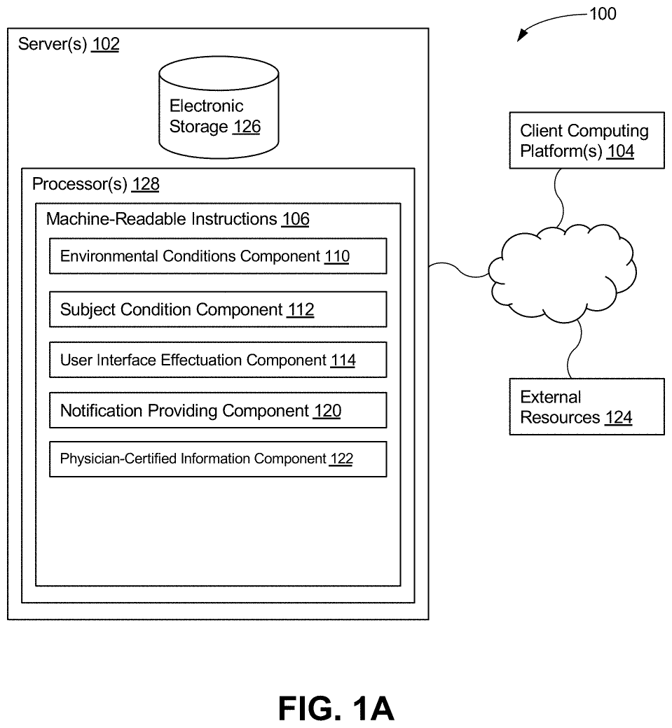

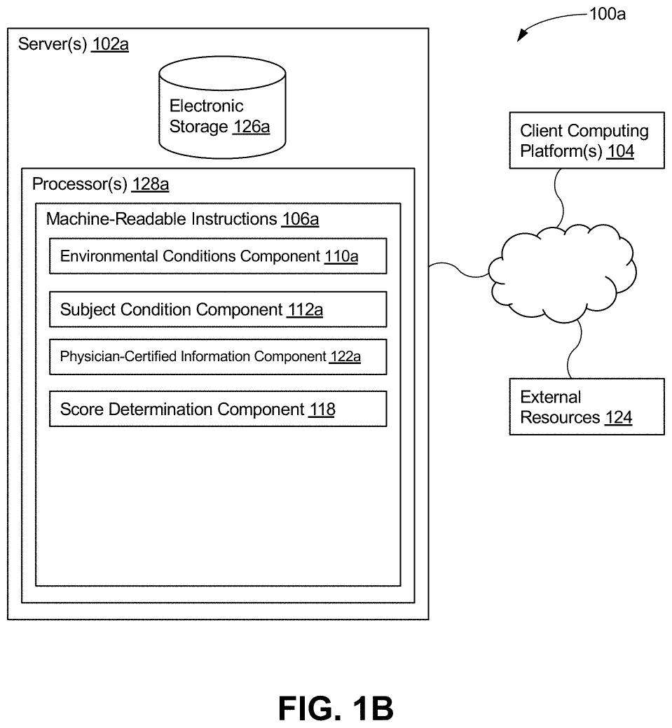

FIG. 1A illustrates a system 100 configured to provide asthma-related information of subjects based on environmental conditions and/or subject condition information, in accordance with one or more implementations. In some implementations, system 100 may include one or more servers 102. Server(s) 102 may be configured to communicate with one or more client computing platforms 104 according to a client/server architecture and/or other architectures. Client computing platform(s) 104 may be configured to communicate with other client computing platforms via server(s) 102 and/or according to a peer-to-peer architecture and/or other architectures. Subjects may access system 100 via client computing platform(s) 104.

Server(s) 102 may be configured by machine-readable instructions 106. Machine-readable instructions 106 may include one or more instruction components. The instruction components may include computer program components. The instruction components may include one or more of environmental conditions component 110, subject condition component 112, user interface effectuation component 114, notification providing component 120, physician-certified information component 122, and/or other instruction components.

Environmental conditions component 110 may be configured to obtain location information related to a physical location of a subject over time. The location information may indicate the physical location of the subject. The physical location of the subject may be of a contextual environment of the subject. The contextual environment may be the immediate space surrounding the subject and of which the subject is working or otherwise breathing in. The physical location may refer to where the subject is over time. The subject may be at, around, or near the physical location for an amount time (e.g., specified by the subject via a graphical user interface presented on client computing platform 104).

The location information may include geographical position system (GPS) coordinates, an internet protocol (IP) address, a geolocation based on client computing platform 104 of the subject and proximate cell towers, and/or other information based on the subject's client computing platform 104 that indicates their physical location. In some implementations, the location information may be obtained in an ongoing manner to ensure accuracy of the physical location indicated. The term “ongoing manner” as used herein may refer to continuing to perform an action (e.g., obtain) periodically (e.g., every 30 seconds, every minute, every hour, etc.) until receipt of an indication to terminate. The indication to terminate may include powering off client computing platform 104, charging one or more of a battery of client computing platform 104, subject input (e.g., via a virtual button), and/or other indications of termination. For example, the subject may move 7 miles from an initial physical location indicated by the location information and the location information should be obtained again to indicate a more accurate physical location of the subject.

In some implementations, the location information may be manually input by the subject to indicate their physical location. For example, manually input location information may include GPS coordinates, a street address, a zip code, a city, and/or other location information. In some implementations, the amount of time, a start time, and/or an end time that the subject is at, around, or near the physical location may be manually input by the subject. For example, the subject may input a start time of 5 PM for 3 hours, or 5 PM-8 PM.

Environmental conditions component 110 may be configured to obtain environmental conditions at or around the physical location indicated by the location information. The environmental conditions may be based on the location information. By way of non-limiting example, the environmental conditions may include values to one or more of pollen levels, weather, an elevation, mold, dust and dander, an air quality index, and/or other environmental conditions. In some implementations, the values may be numerical and associated with units of measure. In some implementations, the values may be non-numerical and generally characterize the respective environmental condition (e.g., good, okay, low, medium, moderate, fair, high, poor, extreme). The values of pollen levels may indicate how much of tree pollen, grass pollen, weed pollen, flower pollen, and/or other pollen is present in air. The values to the weather may include an outdoor temperature, precipitation, humidity, and/or other values indicative of the weather at or around the physical location. The values to the mold may indicate an amount of mold spores in a cubic meter of the air, or other measurement, at or around the physical location. The values of the dust and dander may indicate how much dust and dander from animal fur, hair, feathers, and/or other sources are in an indoor area and/or outdoor area. In some implementations, the values of the dust and dander may be based on the values to the weather and/or other environmental conditions that may exacerbate the dust and dander or may not. The values to the air quality index may indicate how polluted the air at the physical location is. In some implementations, the air may be polluted with the pollen, the dust and dander, the mold spores, and/or other pollutants.

In some implementations, the contextual environment may be indoors (e.g., living room, office). In some implementations, environmental conditions component 110 may be configured to obtain air quality information from one or more digital air purification systems proximate to the subject in the contextual environment, i.e., the indoor area, of the subject. The air quality information may convey ambient air quality measured in the contextual environment of the subject. The digital air purification systems may measure, calculate, or otherwise determine values indicative of quantities of dust and dander, mold, pollen level, and/or other information in the indoors.

In some implementations, the contextual environment may be outdoors (i.e., the subject is outdoors). In some implementations, the air quality information may be obtained from external resources 124. External resources 124 may include satellite systems that collect information about particles in the air, the dust and dander, the mold, the pollen level, and/or other external resources that collect and/or provide air quality information for the outdoors.

In some implementations, the air quality information may be associated with a particular time or period of time. In some implementations, the air quality information may be obtained in an ongoing manner. In some implementations, the air quality information may be obtained at a reoccurring predefined time and may convey the ambient air quality of past times or periods of time.

In some implementations, environmental conditions component 110 may be configured to determine the value to the air quality index based on the air quality information from the one or more digital air purification systems proximate to the subject in the indoor area. Thus, in some implementations, the values to the air quality index may be determined or otherwise derived from the air quality information obtained.

In some implementations, environmental conditions component 110 may be configured to determine the value to the air quality index based on the air quality information obtained from the external resources 124 for the outdoors. Thus, in some implementations, the values to the air quality index may be determined or otherwise derived from the air quality information obtained.

Subject condition component 112 may be configured to obtain subject condition information defining remote diagnostic information, subject health information, asthma management, and/or other information. The subject condition information may include information related to asthma and general health of the subject. The remote diagnostic information may include results to diagnostic tests and medication administrations/consumptions that the subject may conduct himself/herself as opposed to a clinic, doctor's office, or other medical office. The remote diagnostic information may include results of a remote FeNO test, a remote spirometry test, a remote peak flow test, a digital inhaler, and/or other remote diagnostic information. The results may be associated with a date and/or time at which a given test or medication administration occurred. In some implementations, subject condition component 112 may be configured to store the subject condition information to electronic storage 126, a cloud storage, and/or other storage. The subject condition information may be stored to electronic storage 126 indefinitely or for a predefined period of time. The period of time may be defined by a physician, the subject, fixed by system 100, and/or other individual.

A result to the remote FeNO test may determine an amount of lung inflammation of the subject. A result to the remote spirometry test may indicate how much air lungs of the subject is able to breathe in and out and how easily and fast the lungs are able to exhale the air. A result of the remote peak flow test may determine how fast the lungs are able to forcefully exhale after a full inhalation of air. The digital inhaler may indicate correctness of inhalation techniques of medication administered by the digital inhaler, frequency of the administrations, and/or other information related to medication administrations/consumptions via the digital inhaler. For example, the frequency of the administrations may indicate whether the patient is using the digital inhaler enough or too often. For example, the correctness of inhalation techniques may indicate whether the subject is positioning the digital inhaler at a correct angle during administration, shaking the digital inhaler enough or too much prior to use, and/or other correctness of inhalation techniques.

The subject health information may include values for one or more of cardiovascular parameters, sleep parameters, activity parameters, diet parameters, and/or other parameters. The subject health information (i.e., parameter included thereof) may specify measurable, recordable, and/or determined information. The values corresponding to the parameters included in the subject health information may be a particular value, numerical or non-numerical, that characterizes the subject.

By way of non-limiting example, the cardiovascular parameters may include a heart rate, a resting heart rate of the subject, a respiratory rate of the subject, blood pressure of the subject, oxygen saturation of the subject, heart variability, heartbeat strength, heartbeat rhythm, and/or other cardiovascular parameters. By way of non-limiting example, the sleep parameters may include hours of sleep, sleep quality, motion during sleep, breathing patterns during sleep, and/or other sleep parameters. By way of non-limiting example, the activity parameters may include minutes exercised; distance walked, ran, cycled, and/or swam; speed of the walk, the run, the cycle, and the swim; inclination of the walk, the run, and the cycled; acceleration, time standing, and/or other activity parameters. By way of non-limiting example, diet parameters may include caloric intake, weight, weight goal, height, body mass index (BMI), various vitamin intake, food restrictions, food intake, liquid intake, substance intake, and/or other diet parameters. Some of the subject health information may be obtained from one or more wearable devices worn by the subject. For example, the wearable devices may include an orientation sensor, a location sensor, a pressure sensor, a temperature sensor, a light sensor, an audio sensor, cardiovascular sensors, and/or other sensors. For example, the cardiovascular sensors may include an electrodermal activity (EDA), an electrocardiography (EKG or ECG) sensor, a blood volume pulse (BVP) sensor, a respiration sensor, a blood pressure sensor, and/or other cardiovascular sensors. It is to be expressly understood, however, that the subject health information obtained and the sensors that facilitation the obtainment are for the purpose of illustration and description only and are not intended as a definition of the limits of this disclosure.

The asthma management information may include an asthma action plan (AAP) and/or other asthma management information. The AAP may describe a plan for controlling asthma of the subject and asthma treatment guidance for the subject to follow. The AAP may be generated by the subject and a physician. As used herein, the term “physician” may refer to a doctor, a clinical, a nurse, a respiratory specialist, and/or or other authorized medical practitioner. In some implementations, the AAP may be generated based on a known asthma action plan template. In some implementations, the asthma management information may include scores to an asthma control test (ACT). The ACT may refer to questions related to controlling their asthma that the subject may answer/score themselves. In some implementations, the ACT may be a known set of questions. In some implementations, the known set of questions may be altered by the physician. The subject may answer the ACT periodically such as once a week, once a day, subsequent to severe asthma symptoms, and/or other at times.

In some implementations, physician-certified information component 122 may be configured to obtain physician-certified condition information and/or other information. The physician-certified condition information may include results to tests and scores to tests/questionnaires collected by the physician related to the asthma of the subject. The physician-certified condition information may include physician-supervised diagnostic information, physician-administered evaluations of the subject, and/or other information collected by the physician related to the subject.

The physician-supervised diagnostic information may include results to a physician-administered FeNO test, a physician-administered spirometry test, a physician-administered peak flow test, and/or other information. The physician-administered FeNO test, the physician-administered spirometry test, and the physician-administered peak flow test, may be the same as or similar to the remote FeNO test, the remote spirometry test, and the remote peak flow test, respectively, though administered/conducted by the physician. In some implementations, the tests that are physician-administered may be conducted at a medical office, for example, or other setting (e.g., home of subject during house call). In some implementations, the physician-certified condition information may be obtained from clinical systems (e.g., associated with the medical office, hospitals, etc.), laboratory systems, outpatient clinic information technology (IT) systems, and/or other medical information systems, and/or external resources 124.

The physician-administered evaluations may include scores and/or notes to an asthma control questionnaire (ACQ), a physician-administered ACT, and/or other physician-administered evaluations. The scores to the ACQ may measure change in the controlling of the asthma and adequacy of the controlling of the asthma by the subject at a point in time (i.e., how well the subject is following the AAP). The physician may administer or ask questions included in the ACQ to the subject. In some implementations, the ACQ may be generated based on a known asthma control questionnaire template. In some implementations, the ACQ may be generated by the physician. The physician-administered ACT may be similar to the ACT described herein, though administered/asked by the physician as opposed to self-administered/self-completed by the subject. The physician-certified condition information may be stored to cloud storage, electronic storage 126, and/or other storage.

User interface effectuation component 114 may be configured to effectuate presentation of a graphical user interface that conveys the subject condition information and/or other information related to the subject. For example, the other information related to the subject may be stored in electronic storage 126. Presentation of the graphical user interface may be effectuated on client computing platform 104 associated with the subject. In some implementations, the graphical user interface may further convey the physician-certified condition information, the environmental conditions information, the location information, correlations determined between at least the subject condition information and the environmental conditions, the manual subject condition information, notifications for the subject, and/or other information. The graphical user interface may include user interface elements. Individual user interface elements may be configured to be selected by the subject to cause presentation of particular information related to the subject (e.g., the subject condition information). Individual user interface elements may be configured to be selected or utilized by the subject to complete the ACT or other questionnaires. The user interface elements may be configured to facilitate subject interaction with the graphical user interface, subject entry, and/or selection. By way of non-limiting illustration, the user interface elements may include one or more of text input fields, drop down menus, check boxes, display windows, virtual buttons, and/or other user interface elements.

In some implementations, subject condition component 112 may be configured to obtain new results of the FeNO test, the remote spirometry test, the remote peak flow test, the digital inhaler, the physician-administered FeNO test, the physician-administered spirometry test, and the physician-administered peak flow test, and/or other remote diagnostic information, and/or other physician-supervised diagnostic information. The new results may refer to most recently obtained/measured results. In some implementations, subject condition component 112 may be configured to update the remote diagnostic information and physician-supervised diagnostic information with the respective new results. Updating the remote diagnostic information and physician-supervised diagnostic information may include adding the new results, replacing the results with the respective new results, and/or other updates. Thus, the remote diagnostic information and physician-supervised diagnostic information conveyed by the graphical user interface may include their results and the respective new results.

In some implementations, notification providing component 120 may be configured to determine increases or otherwise changes in the environmental conditions. The changes (e.g., increases) in the environmental conditions may indicate asthma triggers of the subject. Asthma triggers may cause activation of asthma symptoms or flare-ups of the subject. By way of non-limiting example, the changes to the environmental conditions may include increases in the pollen levels, the elevation, the mold, the dust and dander, the air quality index, and/or other environmental conditions reaching particular values indicative of the asthma triggers. In some implementations, to indicate the asthma triggers, the changes in the environmental conditions may including all the environmental conditions reaching specific threshold values for the respective environmental condition, all the environmental conditions reaching specific threshold values for the respective environmental condition within a particular amount of time, individual ones of the environmental conditions reaching the respective specific threshold values, individual environmental conditions reaching the respective specific threshold values within a particular amount of time, a particular amount of the environmental conditions reaching the respective specific threshold values, a particular amount of the environmental conditions reaching the respective specific threshold values within a specific amount of time, and/or other changes in the environmental conditions. In some implementations, the threshold values may be of a metric unit, or lack thereof, of the respective environmental condition. The threshold values may be known values that indicate the asthma triggers or may be defined by the physician, the subject, system 100, and/or other individuals. The particular amount of time may be defined by the physician, the subject, system 100, and/or other individuals. The particular amount of the environmental conditions changing may be defined by the physician, the subject, system 100, and/or other individuals.

Responsive to changes in the environmental conditions, notification providing component 120 may be configured to generate notifications. A notification may include the changes in the environmental conditions determined. For example, the notification may be generated responsive to the value of the tree pollen increasing to 18 past a threshold value of 15 and include information related to the increase past the threshold value of 15. User interface effectuation component 114 may be configured to convey the notification to the subject via the graphical user interface presented. In some implementations, the notification may include a pop-up window, a noise, haptics, and/or other notifications. The information conveyed by the graphical user interface (e.g., the subject condition information, the notifications, etc.), may allow for incremental increases in asthma health and reduction in catastrophic medical incidents of the subject.

In some implementations, subject condition component 112 may be configured to determine correlations between at least the subject condition information and the environmental conditions and/or between other information related to the subject and their asthma. That is, for example, a determined increase in elevation may correlate with a particular result to the remote FeNO test. Such correlation may imply that increased or high elevations affect the asthma of the subject as indicated by the particular result to the remote FeNO test. It will be appreciated that the description herein of “correlations” between the subject condition information and changes in environmental conditions which are positively correlated is not intended to be limiting, and that negative correlations the subject condition information and changes in environmental conditions are also contemplated, and may be included in the generic “correlations”. The determination of negative correlations may be made in cases where values of low humidity (i.e., very dry conditions) cause a high result to the remote FeNO test indicative of extreme lung inflammation. The determined correlations may be conveyed to the subject via the graphical user interface.

FIG. 1B illustrates a system 100a configured to provide a respiratory health score of a subject, in accordance with one or more implementations. In some implementations, system 100a may include one or more servers 102a. Server(s) 102a may be configured to communicate with one or more client computing platforms 104 (the same as FIG. 1) according to a client/server architecture and/or other architectures. Client computing platform(s) 104 may be configured to communicate with other client computing platforms via server(s) 102a and/or according to a peer-to-peer architecture and/or other architectures. Subjects may access system 100a via client computing platform(s) 104.

Server(s) 102a may be similar to server(s) 102 as described elsewhere in this disclosure, though included in system 100a. Server(s) 102a may include electronic storage 126a similar to electronic storage 126, though included in server(s) 102a. Server(s) 102a may be configured by machine-readable instructions 106a similar to machine-readable instructions 106 as described elsewhere in this disclosure, though included in server(s) 102a. Machine-readable instructions 106a may include one or more instruction components. The instruction components may include computer program components. The instruction components may include one or more of environmental conditions component 110a, subject condition component 112a, score determination component 118, physician-certified information component 122a, and/or other instruction components. Environmental conditions component 110a, subject condition component 112a, and physician-certified information component 122a may be similar to environmental conditions component 110, subject condition component 112, and physician-certified information component 122, respectively, described elsewhere in this disclosure (for FIG. 1A), though included in server(s) 102a.

In some implementations, some of or all of the instruction components of processor(s) 128 (included in server(s) 102 of FIG. 1A) and some of or all of the instruction components of processor(s) 128a (included in server(s) 102a) may be executed by a single processing unit or the same processing units (e.g., processor(s) 128). The configuration described herein is for illustrative purposes only and is not intended to be limiting.

In some implementations, subject condition component 112a may be configured to receive manual subject condition information from client computing platform 104 associated with the subject described in FIG. 1A. The manual subject condition information may be the same as the subject condition information though manually input by the subject via the graphical user interface presented on their client computing platform 104.

Score determination component 118 may be configured to determine the respiratory health score of the subject based on the environmental conditions, the subject condition information, the manual subject condition information, the physician-certified condition information, and/or other information. In some implementations, determining the respiratory health score based on the manual subject condition information may be different than determining the respiratory health score based on the subject condition information. That is, the manual subject condition information may be given less consideration in the respiratory health score than the subject condition information that is based on the remote diagnostic information obtained (i.e., obtained from respective remote tests) and the subject health information obtained (i.e., from the wearable devices). The respiratory health score may indicate a current respiratory condition of the subject relative to asthma of the subject. The current respiratory condition may characterize lung health, breathing ability, and/or other respiratory conditions of the subject. The respiratory health score may be numerical (e.g., 9 out of 10) or non-numerical (e.g., A, B, very good, fair, etc.).

In some implementations, score determination component 118 may be configured to receive a physician indication to determine a respiratory health score. In some implementations, the physician indication may be received upon the physician selecting a virtual button, for example, included on a graphical user interface presented via client computing platform 104 associated with the physician. The respiratory health score may be determined responsive to receipt of the physician indication. In some implementations, the determination of the respiratory health score may be defined by the physician to reoccur at a particular time. For example, the physician may define for the of the respiratory health score determination to occur weekly every Friday, monthly every 1st day of the month, or other time.

In some implementations, the respiratory health score may be present (e.g., by a component the same as or similar to user interface effectuation component 114 of FIG. 1A) to the subject and/or the physician via client computing platforms 104. In some implementations, the respiratory health score may be included in the graphical user interface of FIG. 1A presented to the subject. In some implementations, the graphical user interface presented via client computing platform 104 associated with the physician may include the respiratory health score.

In some implementations, score determination component 118 may be configured to compare the conveyed information (i.e., the respiratory health score, the subject condition information, the physician-certified condition information, the environmental conditions information, etc.) to asthma population information. The asthma population information may include results, values, and/or respiratory health scores (similar to those of the subject condition information, the physician-certified condition information, the environmental conditions information, etc. related to the subject), for a larger population of other subjects with similar asthma. In some implementations, score determination component 118 may be configured to obtain the asthma population information from external resources 124 (e.g., the clinical systems, the laboratory systems, the outpatient clinic IT systems, etc.) for the comparison. The comparison may be conveyed to the subject and/or the physician via the respective graphical user interfaces. Such comparison may increase the pattern recognition (of controlling the asthma) and pattern management reliability related to the asthma, over time.

In some implementations, based on the respiratory health score presented to the physician, the graphical user interface (presented via client computing platform 104 associated with the physician) may enable the physician to, for example, adjust one or more medications for the subject, add medications, adjust the AAP, adjusting counseling, adjusting goals (related to the asthma management), provide recommendations, and/or provide other information for the subject to properly control their asthma. Similar to the graphical user interface presented to the subject in FIG. 1A, the graphical user interface presented to the physician may include user interface elements that facilitate physician interaction with their graphical user interface, entry, and/or selection by the physician for the adjustments, recommendations, and/or other information for the subject.

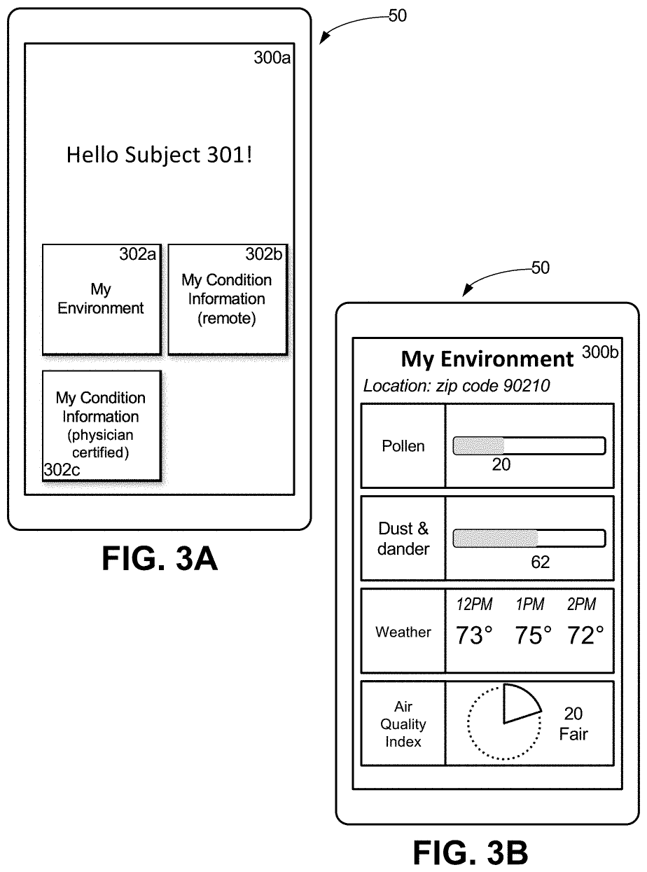

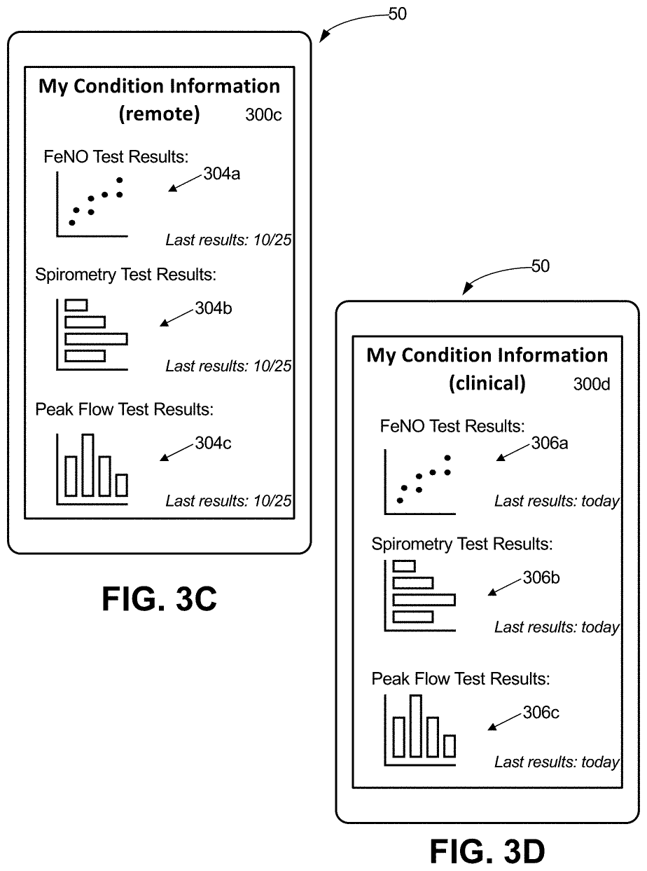

FIG. 3A-D illustrate example implementations, in accordance with one or more implementations. FIG. 3A illustrates a graphical user interface (GUI) 300a on a smartphone 50. GUI 300a may include virtual buttons 302a, 302b, and 302c. Virtual button 302a-c may be selected by a subject 301 to present information related to asthma of subject 301. Virtual button 302a may represent environmental conditions obtained (as described in FIG. 1A-B) of a physical location of subject 301 that may be presented upon selection of virtual button 302a. Virtual button 302b may represent subject condition information (as described in FIG. 1A-B) of subject 301 that may be presented upon selection of virtual button 302b. Virtual button 302c may represent physician-certified condition information (as described in FIG. 1A-B) of subject 301 that may be presented upon selection of virtual button 302c.

FIG. 3B illustrates GUI 300b on smartphone 50, the same as smartphone 50 in FIG. 3A. GUI 300b may present the environmental conditions obtained including values to pollen, dust and dander, weather, and air quality index at the physical location (e.g., zip code 90210) of subject 301 (same as FIG. 3A).

FIG. 3C illustrates GUI 300c on smartphone 50, the same as smartphone 50 in FIG. 3A-B. GUI 300c may present the subject condition information of subject 301. The subject condition information, as described in FIG. 1A-B, may include information related to asthma and general health of the subject. GUI 300c may include results 304a to a remote FeNO test, results 304b to a remote spirometry test, and results 304c to a remote peak flow test that the subject conducted themselves. The results 304a-c included on GUI may include past results and most recent results from date October 25.

FIG. 3D illustrates GUI 300d on smartphone 50, the same as smartphone 50 in FIG. 3A-C. GUI 300d may present the physician-certified condition information of subject 301. The physician-certified condition information, as described in FIG. 1A-B, may include results to tests and scores to tests/questionnaires collected by the physician related to the asthma of the subject. GUI 300c may include results 306a to a physician-administered FeNO test, results 306b to a physician-administered spirometry test, and results 306c to a physician-administered peak flow test. The results 306a-c included on GUI may include past results and most recent results from a date (e.g., today).

In some implementations, server(s) 102, client computing platform(s) 104 (of FIG. 1A-B), and/or external resources 124 (of FIG. 1A-B) may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which server(s) 102, client computing platform(s) 104, and/or external resources 124 may be operatively linked via some other communication media.

A given client computing platform 104 may include one or more processors configured to execute computer program components. The computer program components may be configured to enable an expert or subject associated with the given client computing platform 104 to interface with system 100 and/or external resources 124, and/or provide other functionality attributed herein to client computing platform(s) 104. By way of non-limiting example, the given client computing platform 104 may include one or more of a desktop computer, a laptop computer, a handheld computer, a tablet computing platform, a NetBook, a Smartphone, a gaming console, and/or other computing platforms.

External resources 124 may include sources of information outside of system 100, external entities participating with system 100, and/or other resources. In some implementations, some or all of the functionality attributed herein to external resources 124 may be provided by resources included in system 100.

Server(s) 102 may include electronic storage 126, one or more processors 128, and/or other components. Server(s) 102 may include communication lines, or ports to enable the exchange of information with a network and/or other computing platforms. Illustration of server(s) 102 in FIG. 1 is not intended to be limiting. Server(s) 102 may include a plurality of hardware, software, and/or firmware components operating together to provide the functionality attributed herein to server(s) 102. For example, server(s) 102 may be implemented by a cloud of computing platforms operating together as server(s) 102.

Electronic storage 126 may comprise non-transitory storage media that electronically stores information. The electronic storage media of electronic storage 126 may include one or both of system storage that is provided integrally (i.e., substantially non-removable) with server(s) 102 and/or removable storage that is removably connectable to server(s) 102 via, for example, a port (e.g., a USB port, a firewire port, etc.) or a drive (e.g., a disk drive, etc.). Electronic storage 126 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 126 may include one or more virtual storage resources (e.g., cloud storage, a virtual private network, and/or other virtual storage resources). Electronic storage 126 may store software algorithms, information determined by processor(s) 128, information received from server(s) 102, information received from client computing platform(s) 104, and/or other information that enables server(s) 102 to function as described herein.

Processor(s) 128 may be configured to provide information processing capabilities in server(s) 102. As such, processor(s) 128 may include one or more of a digital processor, an analog processor, a digital circuit designed to process information, an analog circuit designed to process information, a state machine, and/or other mechanisms for electronically processing information. Although processor(s) 128 is shown in FIG. 1 as a single entity, this is for illustrative purposes only. In some implementations, processor(s) 128 may include a plurality of processing units. These processing units may be physically located within the same device, or processor(s) 128 may represent processing functionality of a plurality of devices operating in coordination. Processor(s) 128 may be configured to execute components 110, 112, 114, 118, 120, and/or 122, and/or other components. Processor(s) 128 may be configured to execute components 110, 112, 114, 118, 120, and/or 122, and/or other components by software; hardware; firmware; some combination of software, hardware, and/or firmware; and/or other mechanisms for configuring processing capabilities on processor(s) 128. As used herein, the term “component” may refer to any component or set of components that perform the functionality attributed to the component. This may include one or more physical processors during execution of processor readable instructions, the processor readable instructions, circuitry, hardware, storage media, or any other components.

It should be appreciated that although components 110, 112, 114, 118, 120, and/or 122 are illustrated in FIG. 1 as being implemented within a single processing unit, in implementations in which processor(s) 128 includes multiple processing units, one or more of components 110, 112, 114, 118, 120, and/or 122 may be implemented remotely from the other components. The description of the functionality provided by the different components 110, 112, 114, 118, 120, and/or 122 described below is for illustrative purposes, and is not intended to be limiting, as any of components 110, 112, 114, 118, 120, and/or 122 may provide more or less functionality than is described. For example, one or more of components 110, 112, 114, 118, 120, and/or 122 may be eliminated, and some or all of its functionality may be provided by other ones of components 110, 112, 114, 118, 120, and/or 122. As another example, processor(s) 128 may be configured to execute one or more additional components that may perform some or all of the functionality attributed below to one of components 110, 112, 114, 118, 120, and/or 122.

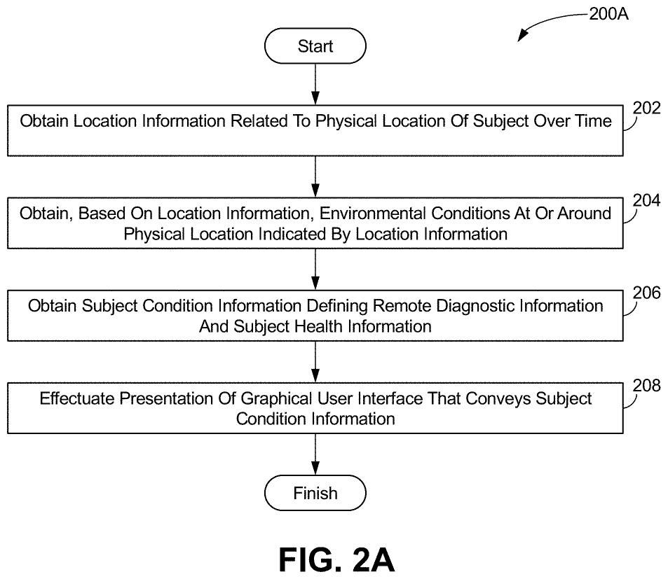

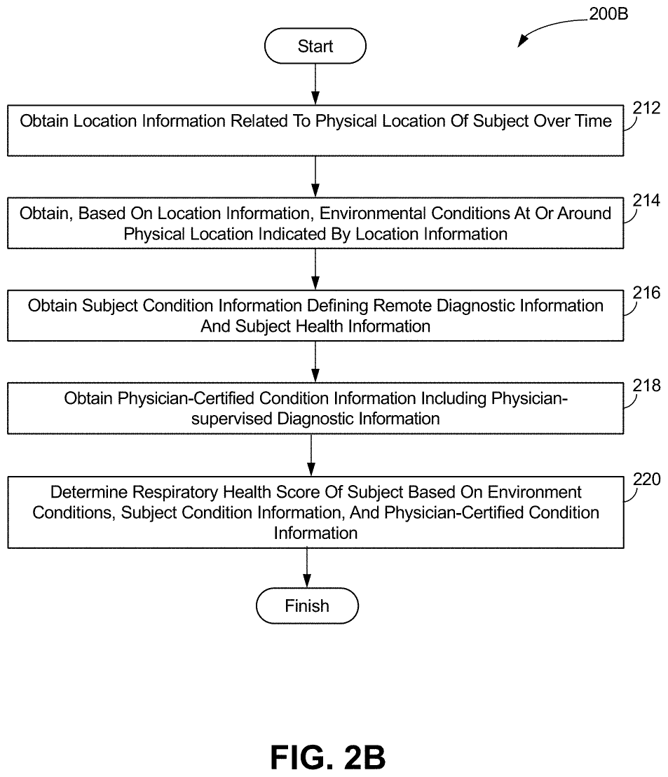

FIG. 2A illustrates a method 200A configured to provide asthma-related information of subjects based on environmental conditions and/or subject condition information, in accordance with one or more implementations. The operations of method 200A presented below are intended to be illustrative. In some implementations, method 200A may be accomplished with one or more additional operations not described, and/or without one or more of the operations discussed. Additionally, the order in which the operations of method 200A are illustrated in FIG. 2A and described below is not intended to be limiting.

In some implementations, method 200A may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200A in response to instructions stored electronically on an electronic storage medium. The one or more processing devices may include one or more devices configured through hardware, firmware, and/or software to be specifically designed for execution of one or more of the operations of method 200A.

An operation 202 may include obtaining location information related to a physical location of a subject over time. The location information may indicate the physical location of the subject. Operation 202 may be performed by one or more hardware processors configured by machine-readable instructions including a component that is the same as or similar to environmental conditions component 110, in accordance with one or more implementations.

An operation 204 may include obtaining, based on the location information, environmental conditions at or around the physical location indicated by the location information. Operation 204 may be performed by one or more hardware processors configured by machine-readable instructions including a component that is the same as or similar to environmental conditions component 110, in accordance with one or more implementations.

An operation 206 may include obtaining subject condition information defining remote diagnostic information subject health information, and/or other information. The remote diagnostic information may include results of a remote fractional exhaled nitric oxide test. Operation 206 may be performed by one or more hardware processors configured by machine-readable instructions including a component that is the same as or similar to subject condition component 112, in accordance with one or more implementations.

An operation 208 may include effectuating presentation of a graphical user interface that conveys the subject condition information and/or other information. Operation 208 may be performed by one or more hardware processors configured by machine-readable instructions including a component that is the same as or similar to user interface effectuation component 114, in accordance with one or more implementations.

FIG. 2B illustrates a method 200B configured to provide asthma-related information of subjects based on environmental conditions and/or subject condition information, in accordance with one or more implementations. The operations of method 200B presented below are intended to be illustrative. In some implementations, method 200B may be accomplished with one or more additional operations not described, and/or without one or more of the operations discussed. Additionally, the order in which the operations of method 200B are illustrated in FIG. 2B and described below is not intended to be limiting.

In some implementations, method 200B may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 200B in response to instructions stored electronically on an electronic storage medium. The one or more processing devices may include one or more devices configured through hardware, firmware, and/or software to be specifically designed for execution of one or more of the operations of method 200B.

An operation 212 may include obtaining location information related to a physical location of a subject over time. The location information may indicate the physical location of the subject. Operation 212 may be performed by one or more hardware processors configured by machine-readable instructions including a component that is the same as or similar to environmental conditions component 110a, in accordance with one or more implementations.

An operation 214 may include obtaining, based on the location information, environmental conditions at or around the physical location indicated by the location information. Operation 214 may be performed by one or more hardware processors configured by machine-readable instructions including a component that is the same as or similar to environmental conditions component 110a, in accordance with one or more implementations.

An operation 216 may include obtaining subject condition information defining remote diagnostic information, subject health information, and/or other information. The remote diagnostic information may include results of a remote fractional exhaled nitric oxide test. Operation 216 may be performed by one or more hardware processors configured by machine-readable instructions including a component that is the same as or similar to subject condition component 112a, in accordance with one or more implementations.

An operation 218 may include obtaining physician-certified condition information and/or other information. The physician-certified condition information may include physician-supervised diagnostic information and/or other information. Operation 218 may be performed by one or more hardware processors configured by machine-readable instructions including a component that is the same as or similar to physician-certified information component 122a, in accordance with one or more implementations.

An operation 220 may include determining a respiratory health score of subject based on the environment conditions, the subject condition information, the physician-certified condition information, and/or other information. The respiratory health score may indicate a current respiratory condition of the subject relative to asthma of the subject. Operation 220 may be performed by one or more hardware processors configured by machine-readable instructions including a component that is the same as or similar to score determination component 118, in accordance with one or more implementations.

Although the present technology has been described in detail for the purpose of illustration based on what is currently considered to be the most practical and preferred implementations, it is to be understood that such detail is solely for that purpose and that the technology is not limited to the disclosed implementations, but, on the contrary, is intended to cover modifications and equivalent arrangements that are within the spirit and scope of the appended claims. For example, it is to be understood that the present technology contemplates that, to the extent possible, one or more features of any implementation can be combined with one or more features of any other implementation.

What is claimed is:

1. A system configured to provide asthma-related information of subjects based on environmental conditions and/or subject condition information, the system comprising:
one or more processors configured by machine-readable instructions to:
obtain location information related to a physical location of a subject over time, wherein the location information indicates the physical location of the subject;
obtain, based on the location information, environmental conditions at or around the physical location indicated by the location information;
obtain subject condition information defining remote diagnostic information and subject health information, wherein the remote diagnostic information include results of a remote fractional exhaled nitric oxide (FeNO) test; and
effectuate presentation of a graphical user interface that conveys the subject condition information.

2. The system of claim 1, wherein the environmental conditions include values to one or more of pollen levels, weather, an elevation, mold, dust and dander, and/or an air quality index, wherein the graphical user interface conveys the environmental conditions.

3. The system of claim 2, wherein the value to the air quality index is based on one or more digital air purification systems proximate to the subject.

4. The system of claim 1, wherein the remote diagnostic information includes results of a remote spirometry test, a remote peak flow test, and a digital inhaler.

5. The system of claim 1, wherein the subject health information includes values for one or more of cardiovascular parameters, sleep parameters, activity parameters, and/or diet parameters, wherein some of the subject health information is obtained from wearable device worn by the subject.

6. The system of claim 1, wherein the subject condition information includes asthma management information, wherein the asthma management information includes an asthma action plan for controlling asthma of the subject and asthma treatment guidance for the subject.

7. The system of claim 4, wherein the one or more processors are further configured by the machine-readable instructions to:
obtain new results of the remote FeNO test, the remote spirometry test, the remote peak flow test, and the digital inhaler; and
update the remote diagnostic information with the new results, wherein the remote diagnostic information conveyed by the graphical user interface include the results and the new results.

8. The system of claim 2, wherein the one or more processors are further configured by the machine-readable instructions to:
determine increases in the environmental conditions; and
generate a notification responsive to the increases in the environmental conditions.

9. The system of claim 8, wherein the increases to the environmental conditions include the pollen levels, the elevation, the mold, the dust and dander, and/or the air quality index reaching particular values indicative of asthma triggers.

10. The system of claim 1, wherein the one or more processors are further configured by the machine-readable instructions to:
obtain physician-certified condition information, wherein the physician-certified condition information includes at least physician-supervised diagnostic information, wherein the physician-supervised diagnostic information includes results to a physician-administered FeNO test, a physician-administered spirometry test, and a physician-administered peak flow test, wherein the graphical user interface conveys the physician-certified condition information.

11. A system configured to provide a respiratory health score of a subject, the system comprising:
one or more processors configured by machine-readable instructions to:
obtain location information related to a physical location of the subject over time, wherein the location information indicates the physical location of the subject;
obtain, based on the location information, environmental conditions of at or around the physical location indicated by the location information;
obtain subject condition information defining remote diagnostic information, and subject health information of the subject, wherein the remote diagnostic information include results of a remote fractional exhaled nitric oxide (FeNO) test;
obtain physician-certified condition information, wherein the physician-certified condition information includes at least physician-supervised diagnostic information, wherein the physician-supervised diagnostic information include results of a physician-administered FeNO test; and
determine the respiratory health score of the subject based on the environmental conditions, the subject condition information, and the physician-certified condition information, wherein the respiratory health score indicates a current respiratory condition of the subject relative to asthma of the subject.

12. The system of claim 11, wherein the physician-supervised diagnostic information includes results to a physician-administered spirometry test and a physician-administered peak flow test.

13. The system of claim 11, wherein the remote diagnostic information includes results to a remote spirometry test, a remote peak flow test, and a digital inhaler.

14. The system of claim 11, wherein the subject health information includes values for one or more of cardiovascular parameters, sleep parameters, activity parameters, and/or diet parameters, wherein some of the subject health information is obtained from wearable device worn by the subject.

15. The system of claim 11, wherein the subject condition information includes asthma management, wherein the asthma management includes an asthma action plan for controlling asthma of the subject and asthma treatment guidance for the subject.

16. The system of claim 15, wherein the physician-certified condition information includes physician-administered evaluations of the subject, wherein the physician-administered evaluations include scores to an asthma control questionnaire that measures change in the controlling of the asthma and adequacy of the controlling of the asthma by the subject at a point in time.

17. The system of claim 11, wherein the one or more processors are further configured by the machine-readable instructions to:
receive a physician indication to determine the respiratory health score, wherein the respiratory health score is determined responsive to receipt of the physician indication.

18. The system of claim 11, wherein the environmental conditions include values to one or more of pollen levels, weather, an elevation, mold, dust and dander, and/or an air quality index.

19. The system of claim 18, wherein the one or more processors are further configured by the machine-readable instructions to:
obtain air quality information from one or more digital air purification systems proximate to the subject at the physical location of the subject, wherein the air quality information conveys the ambient air quality of the physical location of the subject; and
determine the value to the air quality index based on the air quality information.

20. The system of claim 18, wherein the one or more processors are further configured by the machine-readable instructions to:
obtain air quality information from one or more external resources, wherein the air quality information conveys the ambient air quality of the physical location of the subject, wherein the subject is outdoors at the physical location; and
determine the value to the air quality index based on the air quality information.
